(19) United States
(12) Patent Application Publication
(10) Pub. No.: US 2026/0118243 A1
(43) Pub. Date: Apr. 30, 2026

(54) LIGHT SCATTERING FOR DETECTING PARTICLE FORMATION IN FUELS

(71) Applicant: Wyatt Technology, LLC, Goleta, CA (US)

(72) Inventors: Timothy Anderson, Chatham, NJ (US); James Greco, Robbinsville, NJ (US); Hongjiang Liu, Santa Barbara, CA (US); Shivakumar Ramini, Newbury Park, CA (US)

(21) Appl. No.: 19/374,462

(22) Filed: Oct. 30, 2025

Related U.S. Application Data
(60) Provisional application No. 63/713,980, filed on Oct. 30, 2024.

(51) Int. Cl. G01N 15/0205; G01N 33/28

(52) U.S. Cl. CPC G01N 15/0211; G01N 33/2835

(57) ABSTRACT

A system, device, and method for detecting a formation of nanoparticles in a fluid, comprise a temperature controller that provides a temperature of a source of fluid during a temperature ramp cool down operation; a measurement system that detects a particle formation in the source of fluid at an wax particle formation temperature; and an apparatus that detects a cloud point after the particle formation detection by the measurement system and at a temperature less than the wax particle formation temperature, wherein the wax particle formation temperature provides an early warning to users regarding the source of fluid before the fluid becomes visibly cloudy.

### RELATED APPLICATION

This application claims priority to U.S. provisional patent application no. 63/713,980 filed October 30, 2024 and titled “Light Scattering for Detecting Particle Formation in Fuels,” the content of which is incorporated by reference in its entirety.

### FIELD OF THE INVENTION

The disclosed technology generally relates to light scattering instruments. More particularly, the disclosed technology relates to the use of dynamic light scattering systems for detecting sub-visible particulate formation in various fuels.

### BACKGROUND

At relatively cold temperatures, some fuels such as diesel, synthetic, and biofuels can form undesirable waxy crystals causing the fuel to become cloudy. The cloud point is the temperature at which small particles such as wax crystals are first visualized as the fuel is cooled. Measuring the cloud point temperature is an important quality parameter of a fuel, and has been used for decades as a cold-flow test to determine if a fuel is fit for purpose for a climactic region. This is typically in connection with American Society for Testing and Materials (ASTM) protocols such as D2500, D5771, and D5773 by which fuel properties are measured. Here, an instrument may be used that chills the fuel at a set temperature then triggers when the fuel becomes cloudy or turbid. Conventional cloud point testing of fuel materials includes optical or visual interpretations to determine the point at which a fuel has become translucent or cloudy. Such testing is time-consuming, labor intensive, and prone to error.

In addition, cloud point measurements do not provide information about wax particulate material existing in fuels at sub-optical sizes, for example, 0.5nm-2000nm radius. Wax crystals which can grow and become problematic for fuels can form at higher temperatures than the cloud point. Accordingly, the cloud point can provide inaccurately lower cold flow suitability temperatures than may actually be realized. Cold flow additives may further suppress the cloud point. Wax particulates can form in fuels at sub-optical sizes at temperatures higher than the cloud point temperature and over time through various processes (for example, Oswald ripening) can slowly grow and eventually cause a fuel held at these higher temperatures to become turbid or cloudy. The waxy particulates in a fuel can cause wax formation in barges, fuel storage tanks, and vehicle tanks and invariably clog filters or other filter plugging failures. With sub optical information related to the particle size of the wax a user would be able to infer whether filter units may clog with “visually” clear fuels that contain wax particulates at sizes greater than their filter pore sizes. It is desirable to provide the fuel industry with a more accurate and rapid test for wax particle formation, which can lead to a reduction of fuel produce failures.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a system for detecting a formation of nanoparticles in a fluid comprises a temperature controller that provides a temperature of a source of fluid during a temperature ramp cool down operation; a measurement system that detects a particle formation in the source of fluid at an wax particle formation temperature; and an apparatus that detects a cloud point after the particle formation detection by the measurement system and at a temperature less than the wax particle formation temperature, wherein the wax particle formation temperature provides an early warning to users regarding the source of fluid before the fluid becomes visibly cloudy.

In some embodiments, the fluid includes a fuel.

In some embodiments, the particle formation includes an increase in size of nanoparticles of the particle formation during the temperature ramp cool down operation.

In some embodiments, the system measures a decrease in size of nanoparticles of the particle formation during a temperature ramp increase operation.

In some embodiments, system further comprises a plurality of sensors that measure the temperature of the source of fluid.

In some embodiments, the measurement system comprises a light source and a plurality of detectors that measure angles of light emitted from the light source and transmitting through the fluid to characterize the formation of nanoparticles of the fluid.

In some embodiments, the measurement system measures subvisible particles ranging from 0.5 nm to 2000nm.

In some embodiments, the temperature controller provides a constant temperature over a predetermined period of time.

In some embodiments, the temperature controller changes the temperature over a predetermined period of time.

In some embodiments, the apparatus is a forward monitor.

In some embodiments, the measurement system includes at least one of a dynamic light scattering device and a static light scattering device comprising a photodiode.

According to an aspect of the present disclosure, a system for detecting a formation of nanoparticles in a fluid comprises a temperature controller that provides a set temperature of a source of fluid for a predetermined period of time; a monitoring system that performs continuous monitoring of a source of fluid at the set temperature over the predetermined period of time; and a DLS measurement system that detects a formation of particles in the source of fluid at the set temperature during the predetermined period of time, wherein the monitoring system detects a cloud point after the particle formation detection by the DLS measurement system.

According to another aspect of the present disclosure, a method comprises ramping a temperature of a sample (e.g., distillate fuels, renewable diesel (very steep transition point), bio-diesel, or other transparent bio-fuels) from a first temperature (e.g., 10-80 ºC) above a cloud point corresponding to the sample (e.g., 10-50 ºC) below the cloud point at a particular ramp rate (gradually);measuring a hydro-dynamic radius (DLS angle) of the sample during the ramping; and detecting a formation of wax particles during the ramping.

In some embodiments, the hydro-dynamic radius (DLS angle) of the sample is measured by a DLS apparatus that detects a particle formation above a pre-visual cloud point (similar to cloud point) (point where sub-visible wax particles begin to form in solution (sub-micron cloud point))(p/a- per ASTM method), wherein the particle formation includes hard particles that do not change in shape and/or wax particles that form above the cloud point.

In some embodiments, a forward monitor monitors a slight haziness prior to cloud point (measure cloud point) rapidly & accurately as compared to a crude visual inspection.

In some embodiments, the method further comprises pausing the ramping as part of an Oswald ripening process.

According to another aspect of the present disclosure, a method comprises ramping a temperature of a sample (e.g., lubricant, biological feedstock for the lubricant distillate fuels, renewable diesel) from an upper temperature (e.g., 10-80 ºC) above a cloud point corresponding to the sample (e.g., 10-50 ºC) below the cloud point; measuring a hydro-dynamic radius of the sample during the ramping; and detecting the formation of wax particles and characterizing them during the ramping.

According to another aspect of the present disclosure, a method comprises using a DLS to detect wax/hard particle; and using a Laser Monitor/Forward Monitor to monitor the point at which particle size and abundance reaches the point at which the light intensity decreases, and measures a cloud point.

According to another aspect of the present disclosure, an apparatus comprises (reduce thermal mass of system): a metal funnel/isothermal chamber detached from a read head; insulating foam for filling an air gap or air volume above a sample cell; the read head, e.g., t-block, around the sample cell; a metal cap covering in contact with and at the top of the read head to avoid vertical thermal gradient); and a single or multi-stage Peltier or thermoelectric collar to cool the read head and in contact with a heatsink to pull heat away from the Peltier, and an exhaust fan in the vicinity of the heatsink to pull hot air away from Peltier and heatsink.

In some embodiments, the apparatus further comprises a nitrogen gas inlet to dispense cold nitrogen gas around the cuvette according to temperature requirements for auxiliary cooling and to avoid condensation and to assist in cooling.

In another aspect, a system for detecting a formation of nanoparticles in a fluid, comprises a light source that generates a source of light at a fluid sample; a temperature controller that provides a temperature of the fluid sample during a temperature ramp cool down operation; a forward monitor that monitors an intensity of the source of light to indicate a presence of particles in the fluid sample and measures a cloud point; a first photo detector that measures a static light scattering signal when the source of light interacts with the fluid sample; and a second photo detector that measures a dynamic light scattering signal to determine fluctuations in a scattered intensity when the particles scatter the source of light; a measurement system that detects a particle formation in the source of fluid at a wax particle formation temperature, wherein a combination of the first photo detector and the second photo detector detect a presence of the particles before the cloud point is reached.

In some embodiments, the particle formation includes an increase in size of nanoparticles of the particle formation during the temperature ramp cool down operation.

In some embodiments, the system measures a decrease in size of nanoparticles of the particle formation during a temperature ramp increase operation.

### BRIEF DESCRIPTION OF THE DRAWINGS

The above and further advantages of this invention may be better understood by referring to the following description in conjunction with the accompanying drawings, in which like numerals indicate like structural elements and features in the various figures. For clarity, not every element may be labeled in every figure. The drawings are not necessarily to scale, emphasis instead being placed upon illustrating the principles of the invention.

FIG. 1 is a block diagram showing certain components of a cloud point monitoring system, in accordance with some embodiments.

FIG. 2A is a flow diagram of a method for detecting nanoparticle formation in a fuel, in accordance with some embodiments.

FIG. 2B is a flow diagram of another method for detecting nanoparticle formation in a fuel, in accordance with some embodiments.

FIG. 3 is a graph illustrating a dynamic light scattering (DLS) particle formation detection, in accordance with some embodiments.

FIG. 4 is a graph illustrating wax accumulation during an Oswald ripening process, in accordance with some embodiments.

FIG. 5 is a graph of large particles along a DLS temperature range as monitored by a forward monitor, in accordance with some embodiments.

FIG. 6 is a cross-sectional view of an apparatus that detects nanoparticle formation in fuels, in accordance with some embodiments.

FIGS. 7A and 7B are block diagrams of a cloud point monitoring system, in accordance with other embodiments.

FIG. 8 is a graph illustrating particle formation detection at a cloud point monitoring system, in accordance with some embodiments.

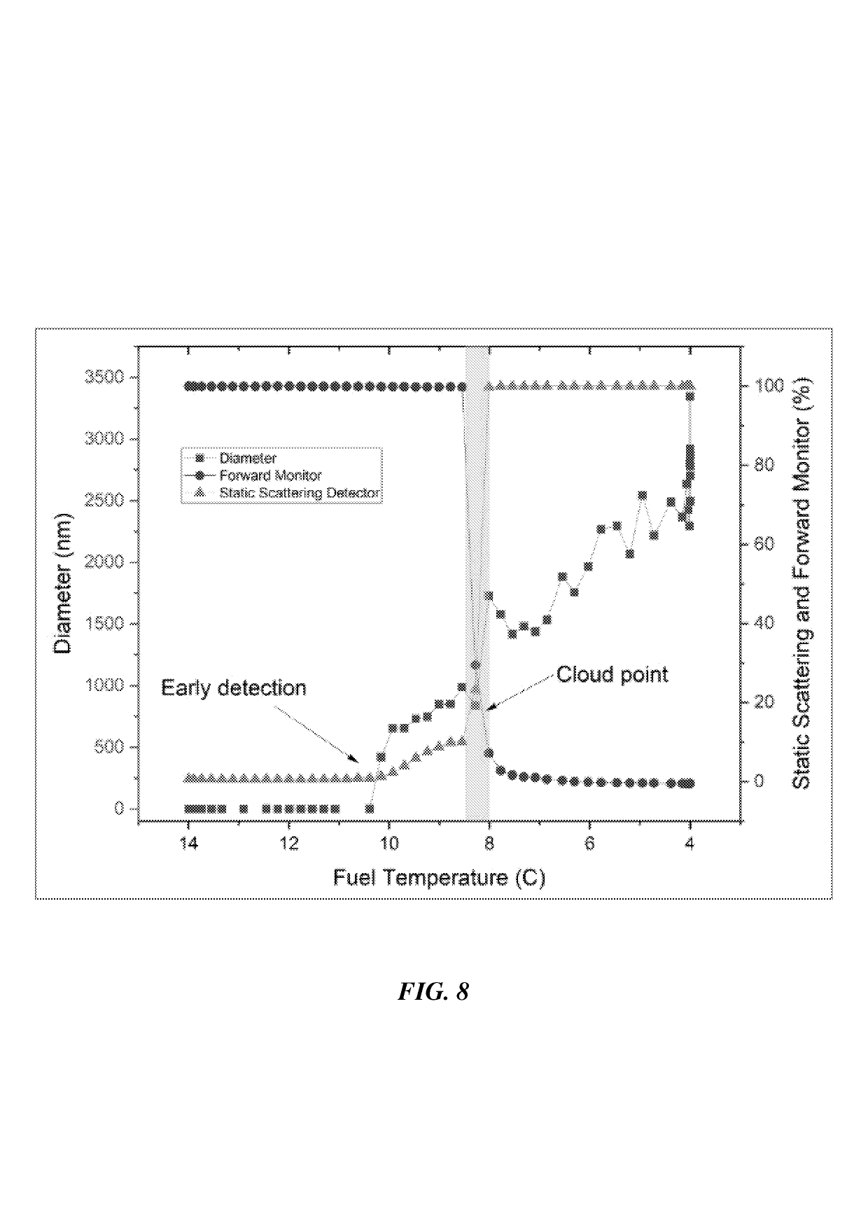

### DETAILED DESCRIPTION

Reference in the specification to an embodiment or example means that a particular feature, structure or characteristic described in connection with the embodiment or example is included in at least one embodiment or example of the teaching. References to a particular embodiment or example within the specification do not necessarily all refer to the same embodiment or example.

The present teaching will now be described in detail with reference to exemplary embodiments or examples thereof as shown in the accompanying drawings. While the present teaching is described in conjunction with various embodiments and examples, it is not intended that the present teaching be limited to such embodiments and examples. On the contrary, the present teaching encompasses various alternatives, modifications, and equivalents, as will be appreciated by those of skill in the art. Moreover, features illustrated or described for one embodiment or example may be combined with features for one or more other embodiments or examples. Those of ordinary skill having access to the teaching herein will recognize additional implementations, modifications, and embodiments, as well as other fields of use, which are within the scope of the present disclosure as described herein.

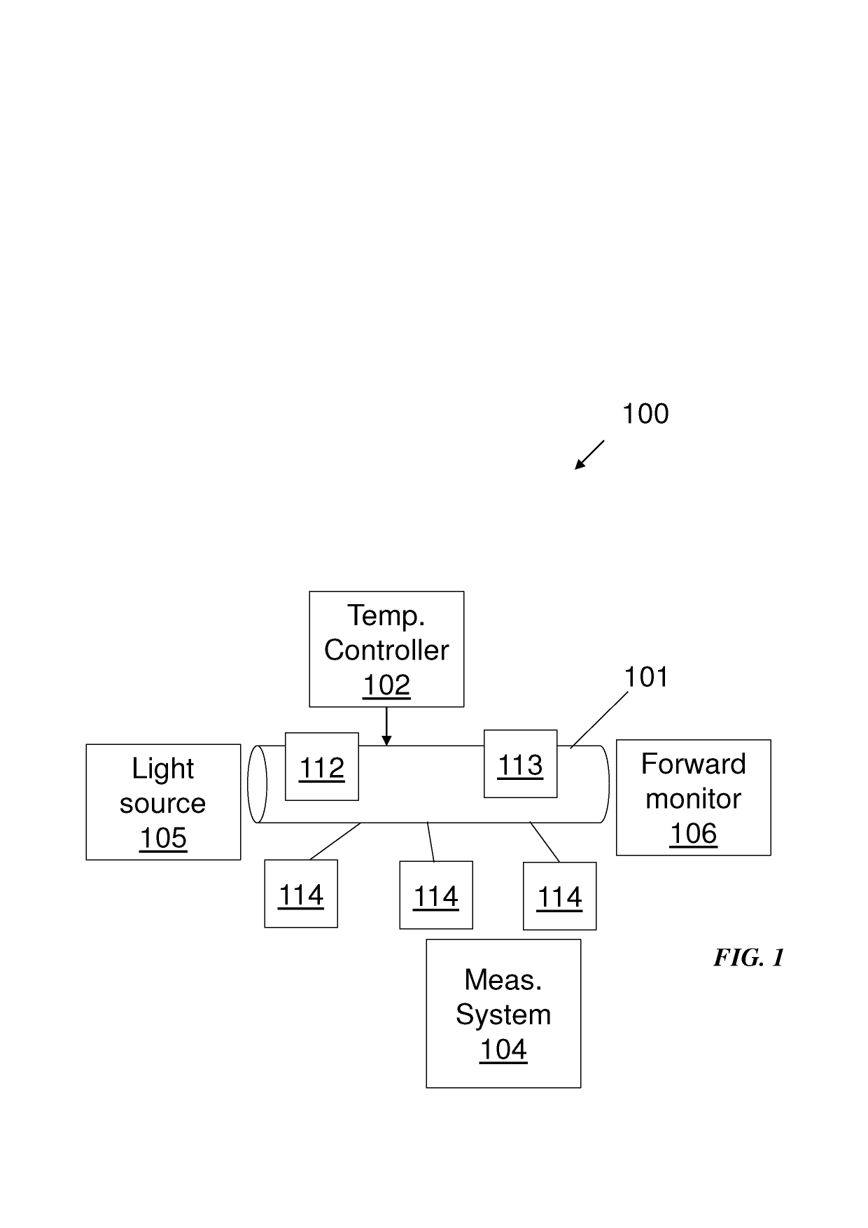

FIG. 1 is a block diagram showing certain components of a cloud point monitoring system 100, in accordance with some embodiments. In some embodiments, the cloud point monitoring system 100 includes a temperature controller 102, a DLS measurement system 104, and a forward monitor detector 106.

The temperature controller 102 can be used to control and measure a wax particle formation temperature of neat petroleum distillates, biofuels, and pyrolyzed waste plastic distillate blends comprised of each variation of these fuels. The wax particle formation temperature provides a more conservative cold flow measurement at higher temperatures than when the fuel becomes visibly cloudy. Neat distillate fuels may include diesel fuel, marine distillate fuel, home heating oil, and the like. Biofuels may include biodiesel, renewable diesel (hydrogenation derived renewable diesel, and other renewable fuels distilled or produced from renewable feedstocks. Pyrolyzed waste plastic distillates may include distillate fractions or the like. In some embodiments, the temperature controller 102 can cool a cuvette 101 or related sample reservoir, and the fuel therein, and more specifically, perform a temperature ramp cool down of a fuel sample to match a specific temperature ramp rate for example, an ASTM D5773 technique describes a proper ramp temperature and describes a fuel’s suitability for low temperature operation and its potential to block fuel filters. The temperature controller 102 can cool a cuvette 101 or related sample reservoir, and the fuel therein, and more specifically, perform a temperature ramp cool down of a fuel sample at a specified temperature ramp rate and then perform a cold soak at a static temperature for a variable length of time, for example, mimicking the 1^{ο}C 16 hour cold soak step for section 3.4 of the CAN/CGSB-3.0 No. 142.0-2019 under “Methods of testing petroleum and associated products : cold soak filter blocking tendency of biodiesel (B100),” incorporated by reference herein, which describes a step causing wax formation through a cold soak step, such as ASTM D2068. For example, it is important how fast and long it takes to hold a fuel at with respect to the wax formation in the fuel. ASTM D5773 for example describes a specific step time with respect to the rate at which it decreases. The system provides a cloud point similar to ASTM but applies techniques performed corresponding devices such as a combination of forward monitors and/or light scattering systems for determining the turbidity of the fuel. Here, a broad range of temperature hold capabilities can be identified then a cold soak technique such as an ASTM-compliant method can be compared, noting that ASTM may be similar but not exact, so that the system can replicate the same profile and use light scattering to monitor the sample. When referring to a cloud point temperature, this is the point where the fluid sample becomes hazy as distinguished from where it matches the ASTM cloud point. The temperature controller 102 includes cooling elements that that can control a temperature of a cuvette 101 or other apparatus holding a fuel sample to range between -50ºC and 120 ºC. Accordingly, a cool down operation performed on a fuel sample can reduce the temperature up to -50ºC, or colder. A temperature sensor 112 is p at the cuvette 101 can fluidly communicate with the fuel within the cuvette 101 and in doing so collect temperature information used by the temperature controller 102 to control the temperature ramp cool down. In doing so, the sensor 112 may provide signals to the controller 102 indicating a current temperature, and in response the controller 102 can determine whether the current temperature is within a predetermined range, such as a cool down range.

A light source 105 is in communication with the cuvette 101 to transmit a source of light to the fuel in the cuvette 101. The light source 105 can comprise any device such as a laser, light emitting diode (LED), and so on that can transmit visible light or non-visible light. In some embodiments, the light source 105 is at one end of the cuvette 101 to transmit a focused or coherent light beam, through at a least a portion of a length of the cuvette 101 in response to receiving a signal from a controller, which may be part of or include the temperature controller 102. The controller controlling the light source 105 may also be in electrical communication with one or more sensors of the system 100, for example, light sensor, temperature sensor, and so on. In some embodiments, the controller comprises at least one processor and memory for storing and processing data generated by the controller.

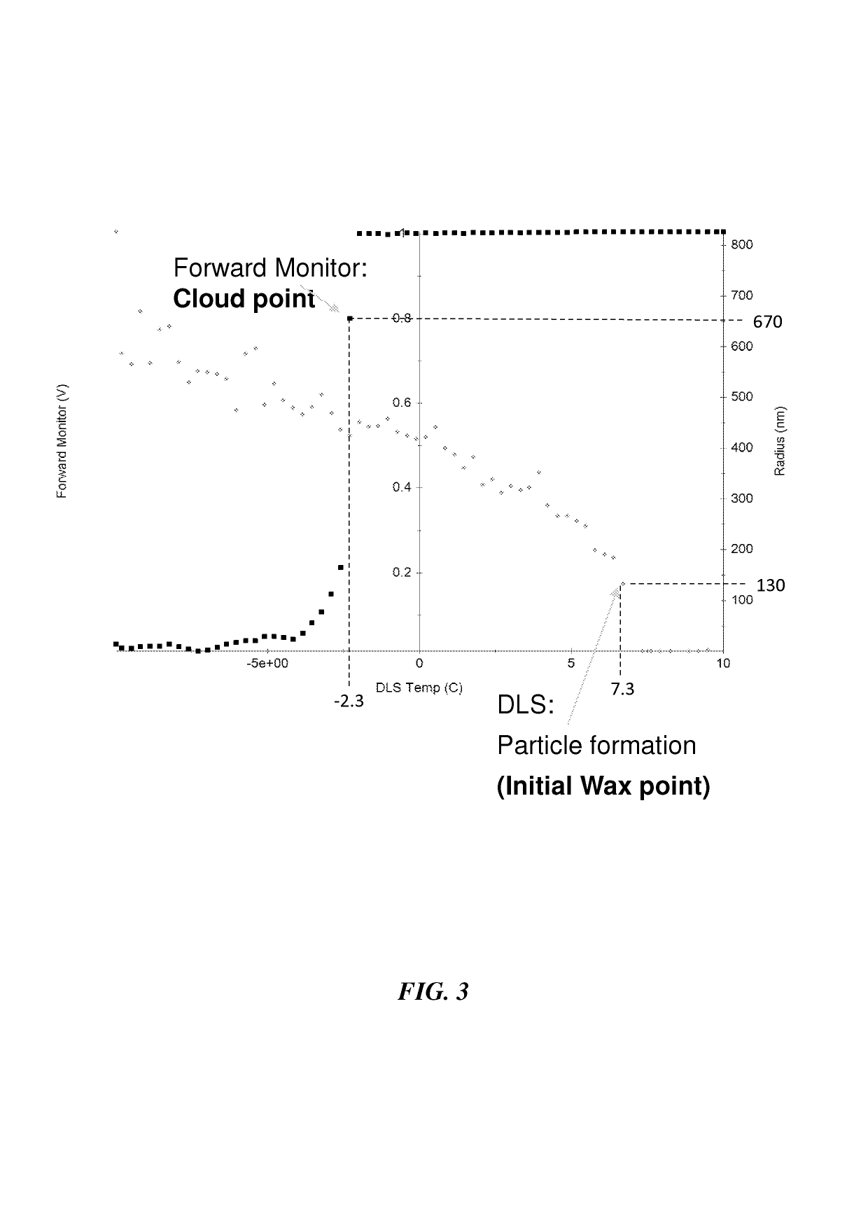

The DLS measurement system 104 operates to characterize the formation of nanoparticles as a fuel sample forms a wax, for example, during temperature deviations caused by the temperature controller 102. Such nanoparticles may be as large as 2000nm, but not limited thereto. For example, if the temperature changes where particles start to be formed, for example, 5-10 ºC above the cloud point, or at higher temperatures, after 1-7 days, the system can analyze whether the particles increase in size or other quantitative information used for characterizing the formation of the particles. For example, shown in FIG. 3, that particle formation occurs and increases in size as the temperature decreases. In some embodiments, the DLS measurement system 104 detects particles, e.g., hard, soft, wax, etc. formation above a pre-visual cloud point, or a point where the particles begin to solidify or freeze at a sub-micron level. Such hard or soft particles can be detected by the DLS measurement system 104 regardless of temperature, and in some cases may be present as an impurity in a sample of interest Comparisons may be made to an ASTM technique when establishing the cloud point. In some embodiments, the DLS measurement system 104 can perform side-scattering operations and operate on a fuel sample received from the temperature controller 102 and takes the scattering angle as detected by detectors 114 into consideration when detecting small particles in the fuel sample. The detectors 114 can be placed at multiple angles relative to the illuminating beam for measuring the light at different angles. Additional detectors monitor the beam intensity and transmission through the cuvette 101. The DLS measurement system 104 operates in connection with the temperature controller 102 in that as the controller 102 causes the temperature of the fuel in the cuvette 101 to approach the cloud point temperature during temperature ramp cool down such that the wax particulates that are formed can be measured by the DLS measurement system 104, and more specifically, scattering the light beam thereby reducing the intensity of the light reflected back to a light sensor 113 positioned at the cuvette 101. It is well-known that for very small particles the scattering is equal at all angles, so any angle may be acceptable. Therefore, not only are the wax particulates measured at the cloud point temperature, but also at temperatures higher than the cloud point temperature such as the wax particle formation temperature where the particulates are minute and at sub-optical sizes.

The DLS measurement system 104 when in operation can provide insights on subvisible particles from ~0.5 nm up to 2µm or 2000nm in radius and can determine the very earliest formation of wax particles when ramping high to low temperatures. The point at which wax first forms can be referred to as the wax particle formation temperature. The wax particle formation temperature is higher than a cloud point onset temperature, or the initial temperature at which the forward monitor detector 106 starts to dim, or detects less light due to the solution becoming saturated with particles resulting in turbidity of the fuel. Therefore, the DLS measurement system 104 monitoring the wax particle formation temperature provides a significantly improved cold flow attribute prediction where wax is determined before it is visible and a temperature at which where fuels have a propensity to eventually cloud when held at a static temperature.

The dynamic light scattering provided by the DLS measurement system 104 can provide further insights into fuels by scanning for the wax particle formation temperature, and a temperature of interest can be held at a granularity of 0.1ºC. By holding at this temperature, observations can be determined as to whether the wax in the fuel will remain at its current particle size or continue to grow, and predictions can be made as to whether they may grow and eventually become visible and cause filter failures well before the fuel "cloudiness" is visible by heritage cloud point measurements. In another example, with the DLS measurement system 104 and its feature of characterizing particle size, it may be used to model the filter blocking tendency (FBT) and determine if a fuel’s propensity to plug filters with a cold flow measurement rather than having to use a large amount of fuel and time measuring it directly over a filter.

The forward monitor detector 106 when utilized in alignment with the cloud point specification can monitor the cloud point. The forward monitor detector 106 can coexist with the measurement system 104 as part of a DLS instrument, and in doing so can be deployed to monitor the dimming of light due to the increase in size and abundance of wax particulates of a sample in the cuvette 101. As the sample temperature ramps from high to low by the temperature controller 102, the sample stays transparent until enough waxing has appeared, which blocks the light source 105, e.g., laser or the like from reaching the forward monitor detector 106, which monitors transmission through the cuvette 101 and can monitor if any clouding of the sample in the cuvette 101 blocks light from the light source 105 from passing through the cuvette 101. Decreases in transmission are measured by the forward monitor detector 106 to calculate the loss of laser intensity in the cuvette 101. In some embodiments, the cloud point can be the temperature at the midway point between the maximum forward monitor and minimal forward monitor. In other embodiments, the cloud point can be measured at any location. For example, as shown in FIG. 3, the forward monitor detector 106 detects no light passing through the cuvette at 0% energy and range up to 100% (shown in the graph as “1” where the black dots are shown in a linear path) where the forward monitor detector 106 detects a maximum amount of light. There is a transition point at the dashed line, e.g., at 2.3 degreesC where the black dots illustrate a dramatic drop to 0.2, or 20% of the light is detected by the forward monitor. The onset temperature can be the initial temperature at which the forward monitor detector 106 detects light in a source of fuel having the onset of wax particle formation and starts to dim. This method offers greater precision than the visual inspection of wax formation. The method in accordance with inventive concepts can be performed where results indicating the presence or increase in wax particles can be determined within a 30-minute time scale or less, compared to hours or even days for a rugged traditional visual inspection method.

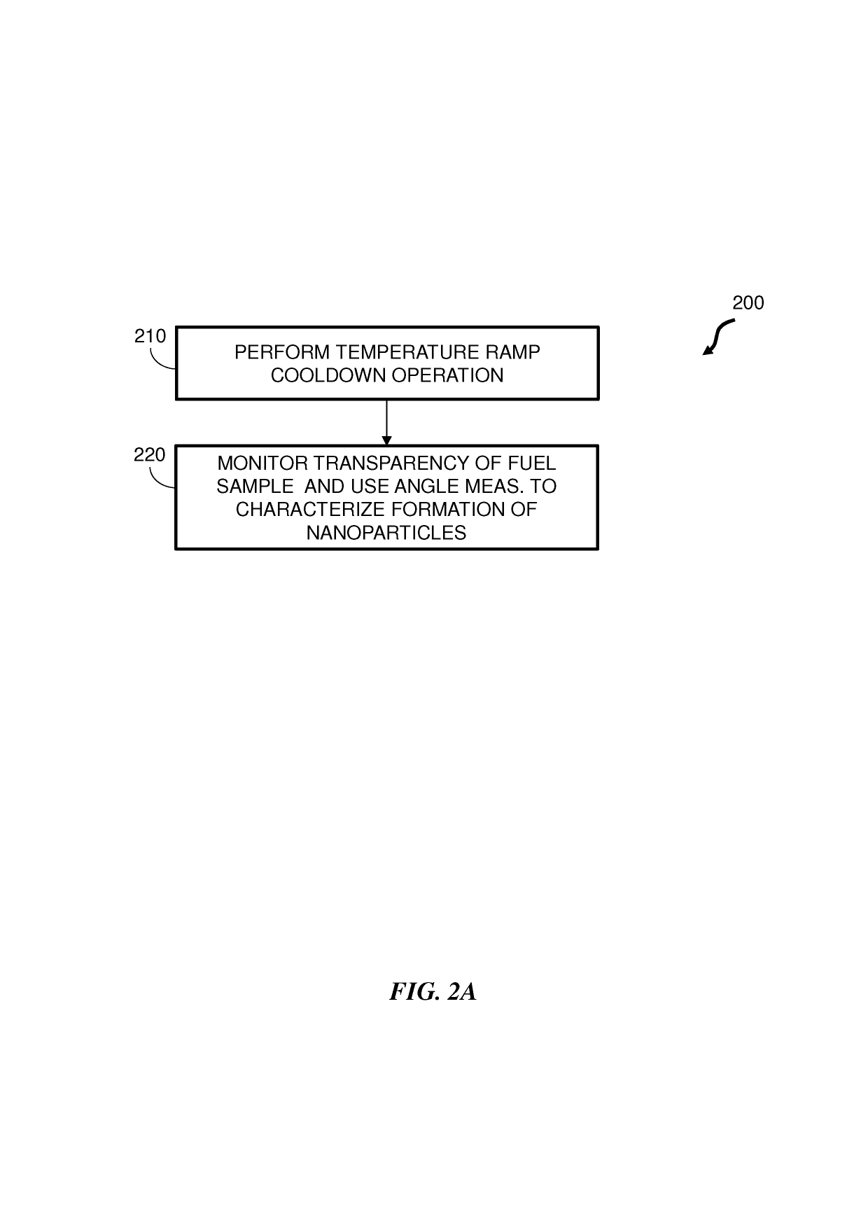

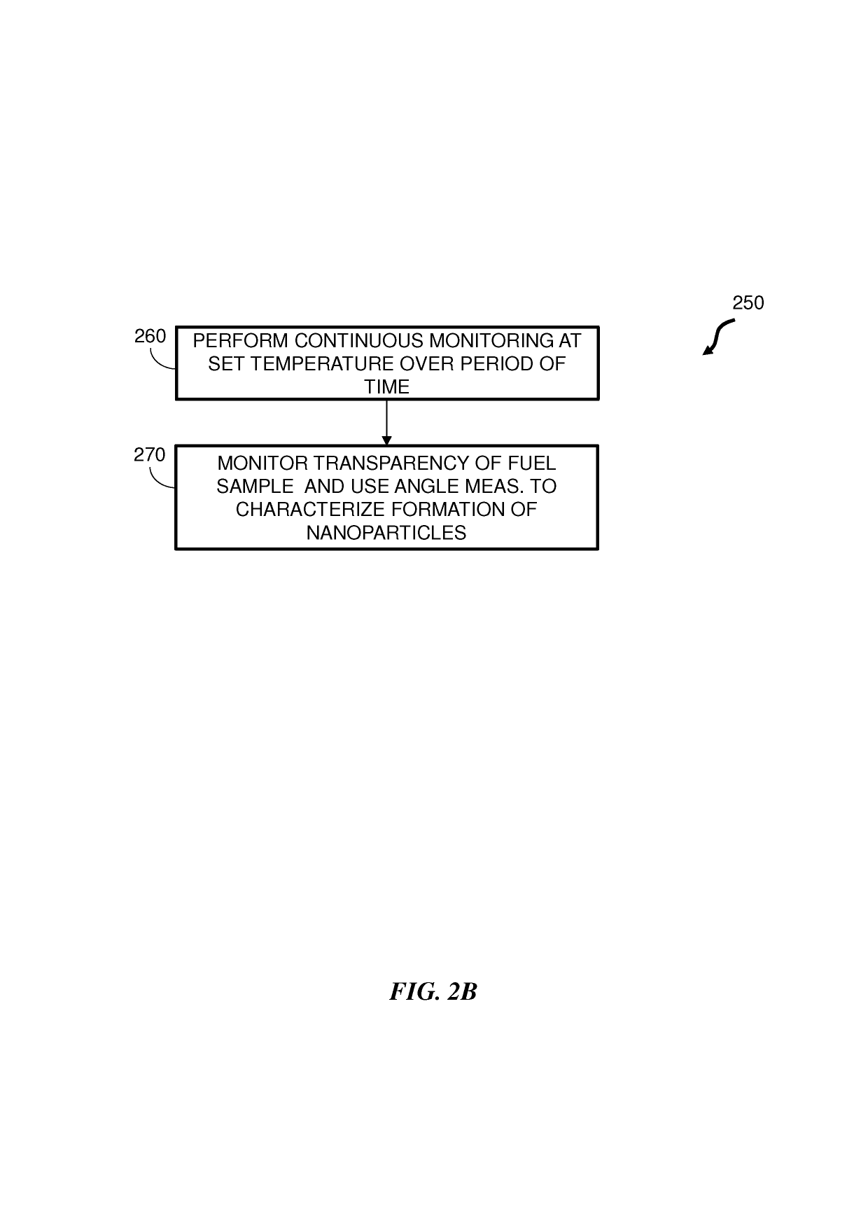

FIG. 2A is a flow diagram of a method 200 for detecting nanoparticle formation in a fuel, in accordance with some embodiments. In describing the method 200, reference is made to components of FIG. 1.

At step 210, the temperature controller 102 performs a temperature cool down operation on a sample of fuel until the fuel enters a transition state where particles begin to form but the fuel is not yet visibly cloudy. The cool down may continue until the fuel becomes visibly cloudy. In some embodiments, the temperature controller 102 can perform a temperature ramp cool down operation to match or exceed the ramp rate of an ASTM cold soak method, for example, in accordance with ASTM D5773 but not limited thereto. In some embodiments, the ramp rate can be flexible, i.e., greater than the ASTM specific ramp rate or less than the ASTM specific ramp rate.

At step 220, the DLS measurement system 104 performs a light scattering operation, and in doing so, uses the DLS angle detected by detectors 114 to characterize the formation of nanoparticles of the fuel sample as wax particles begin to form in a transition state as approaching the cloud point. For example, nanoparticle characterization may include monitoring the formation of particles causing turbidity and determining size increases, number of particles, etc. in the fuel during formation caused by temperature fluctuations. Contemporaneously, the forward monitor detector 106 when utilized in alignment with the cloud point specification can monitor the cloud point. The forward monitor detector 106 monitors the light interacting with the fluid in the cuvette 101, and in particular monitors the amount of light to show the amount of light transmitted through the cuvette 101. In doing so, in some embodiments, the forward monitor can measure the dimming of light or amount of light blocked from reaching the forward monitor detector 106, due to the increase in size and abundance of wax particulates in the sample in the cuvette 101. In other embodiments, in cases where there is a hold, there may be an increase in light observed, or brightening, detected by the forward monitor detector 106.

FIG. 2B is a flow diagram of a method 250 for detecting nanoparticle formation in a fuel, in accordance with some embodiments. In describing the method 250, reference is made to components of FIG. 1.

At step 260, continuous monitoring is performed at a set temperature over a predetermined period of time. Here, the system can hold the temperature of the fluid in the cuvette 101 at a specific temperature and use the forward monitor detector 106 and DLS measurement system 104, for example, to determine particle size of wax particulates formed, etc. during the period of time. If formation of particles increase in size of particles, and the cloud point, e.g., ASTM cloud point, has been met due to decrease in light intensity by forward monitor, is detected after an extended period of time, for example, after 2 days of continuous monitoring (step 270), then this can provide valuable analysis data. The continuous monitoring (step 270) may be performed by the measurement device 104, which may include a DLS system (FIG. 1) or a combination of DLS and SLS systems (FIG. 11). In some embodiments, the temperature can be changed, i.e., increased or decreased, during this predetermined period of time.

FIG. 3 is a graph illustrating a dynamic light scattering (DLS) particle formation detection, in accordance with some embodiments. In particular, FIG. 3 illustrates a DLS measurement system 104 detecting particle formation before the cloud point, e.g., ASTM cloud point. In other words, wax particles may be formed before they can be visually observed since the fuel in this state is not yet cloudy or turbid. An instrument can be used to chill the fuel at a predetermined rate and trigger when the fuel forms particles or becomes cloudy or turbid. In the example graph shown in FIG. 3, the fuel is a renewal diesel blend. However, other liquids such as fuels or the like may equally apply. Here, the temperature controller 102 slowly ramps the temperature in the cuvette or other holder of a fuel sample from +10ºC to -10ºC at 0.2ºC/minute. The DLS measurement system 104 determines that particle formation starts at approximately +7.3 ºC, which is well before the cloud point temperature (-2.3ºC). During the temperature cool down, particles can grow significantly, for example, having a radius that increases from 130 nm to 670nm.

The initial wax point where the DLS detects particle formation may provide an early warning to users regarding problematic fuels before the fuels become visibly cloudy. In addition, the quantitative monitoring of particles may help users make better filter decisions. For example, as described above, if a user knows the size of the particles, the user can make informed decisions as to whether filters or systems may become clogged or fouled based on knowledge of the pore size of their filters. A fuel may be clear, but if it generates particles and is not at a sufficient temperature (depending on the type of fuel, etc.), the fuel although clear, may nevertheless contain particles that could be filtered out and clog certain filters. It is not uncommon for waxes to be heated much higher than their wax point to melt due to their crystallization energies.

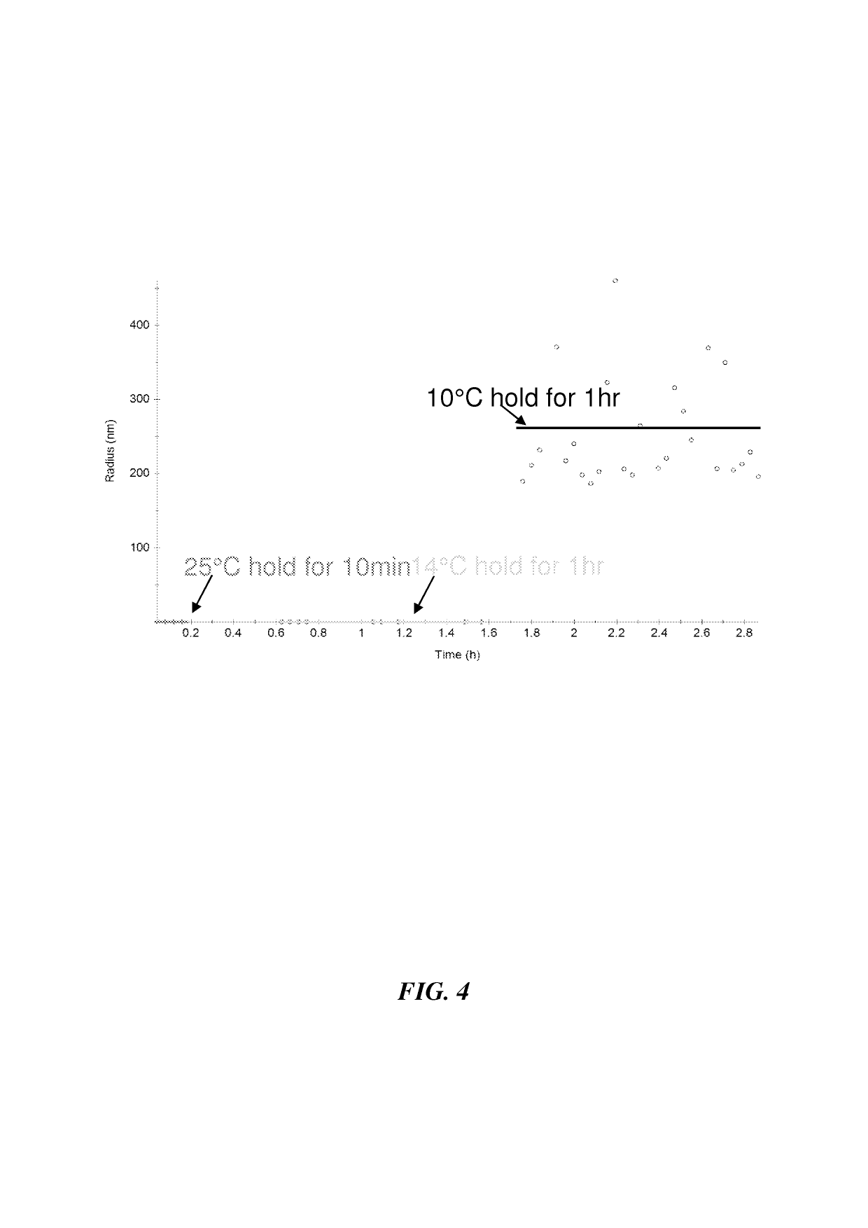

FIG. 4 is a graph illustrating wax accumulation during an Oswald ripening process, in accordance with some embodiments.

As described above, wax particulates can form in fuels at sub-optical sizes at temperatures higher than the cloud point, e.g., ASTM, and over time through various processes, such as an Oswald ripening process, where small crystals may dissolve and redeposit onto large crystal, which can result in wax particle aggregation. As shown in FIG. 4, wax seeds can form immediately at approximately 10ºC, or higher than the cloud point. An early on-set wax seed may have a radius of about 252nm +/- 71nm. The temperature controller 102 may hold the temperature steady above the cloud point temp, e.g., 10ºC. For example, FIG. 4 illustrates 10ºC and 25ºC hold temperatures to show that a user could hold the temperature at a certain point and that no particles would form because the energy of the system is too high for wax to form. However, at a certain point when the temperature has decreased, the energy of the system can be reduced to the point where, when held, wax particles can begin to form. The temperature controller 102 may hold, or maintain, the temperature for a sufficiently long time, for example, 1 hour or more so that particle size growth can be observed to determine if the fuel becomes visibly cloudy or understand the growth properties of the wax particles. More specifically, a hold study can show whether the particles stay the same size or increase in size over an extended period. If the temperature remains steady in this manner, this wax appearance test can assist users, for example, storing fuels in refineries, terminals, etc. to better understand the storage conditions for their fuels.

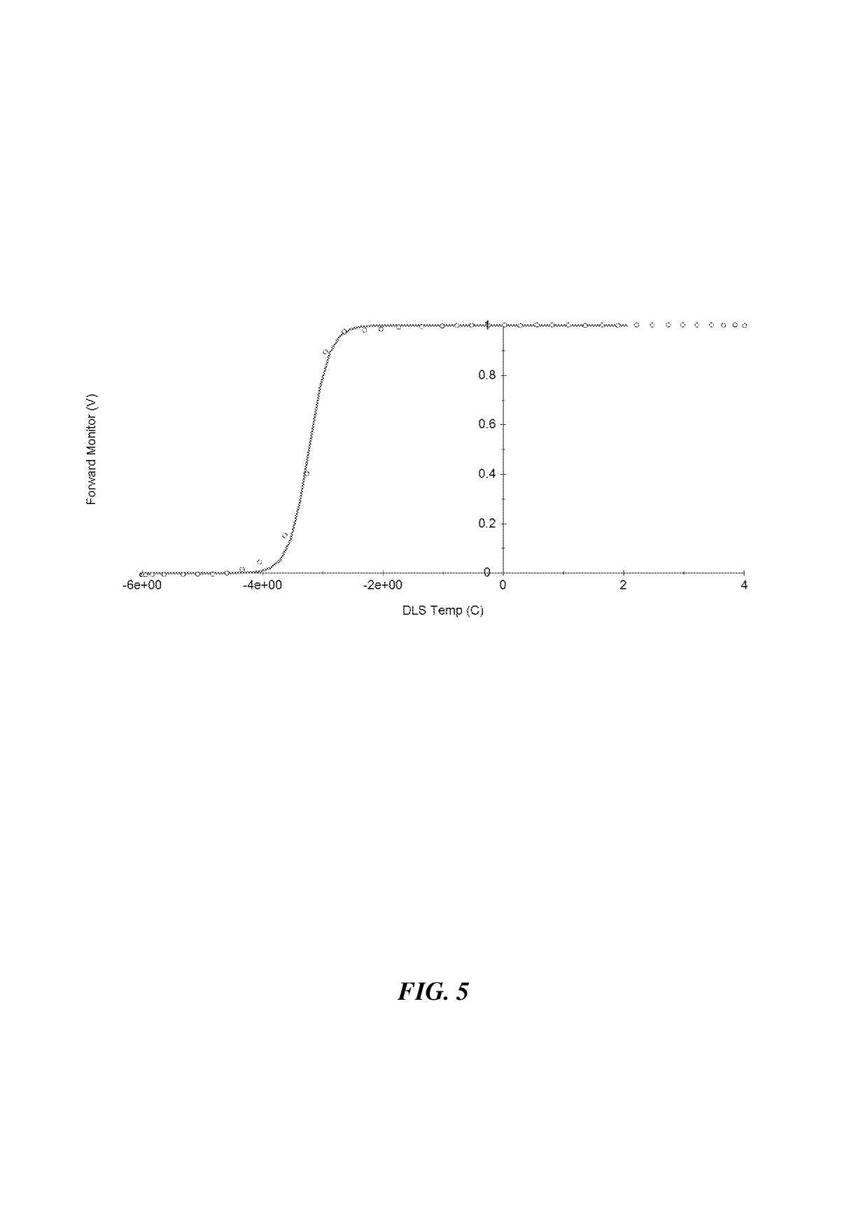

FIG. 5 is a graph of large particles along a DLS temperature range as monitored by a forward monitor, in accordance with some embodiments.

Here, a sample transition temperature of around -3.2 ºC is shown with respect to a cloud point including large particles in a fuel sample. In some embodiments, the temperature ramp may range from 14ºC to -6ºC, for example, at 0.5 ºC/min. In other embodiments, the temperature ramp may range from 10ºC to -10ºC, for example, at 0.5 ºC/min. Other ranges may equally apply. In another example (not shown), a sample is cooled down to the cloud point, held, and then heated up to interrogate the temperature at which the wax particles fully melt. This illustrates an inventive feature in that the system and method in accordance with embodiments herein offer more analytical detail than human visual confirmation. Also, the forward monitor used herein is unaffected by the presence of the particles.

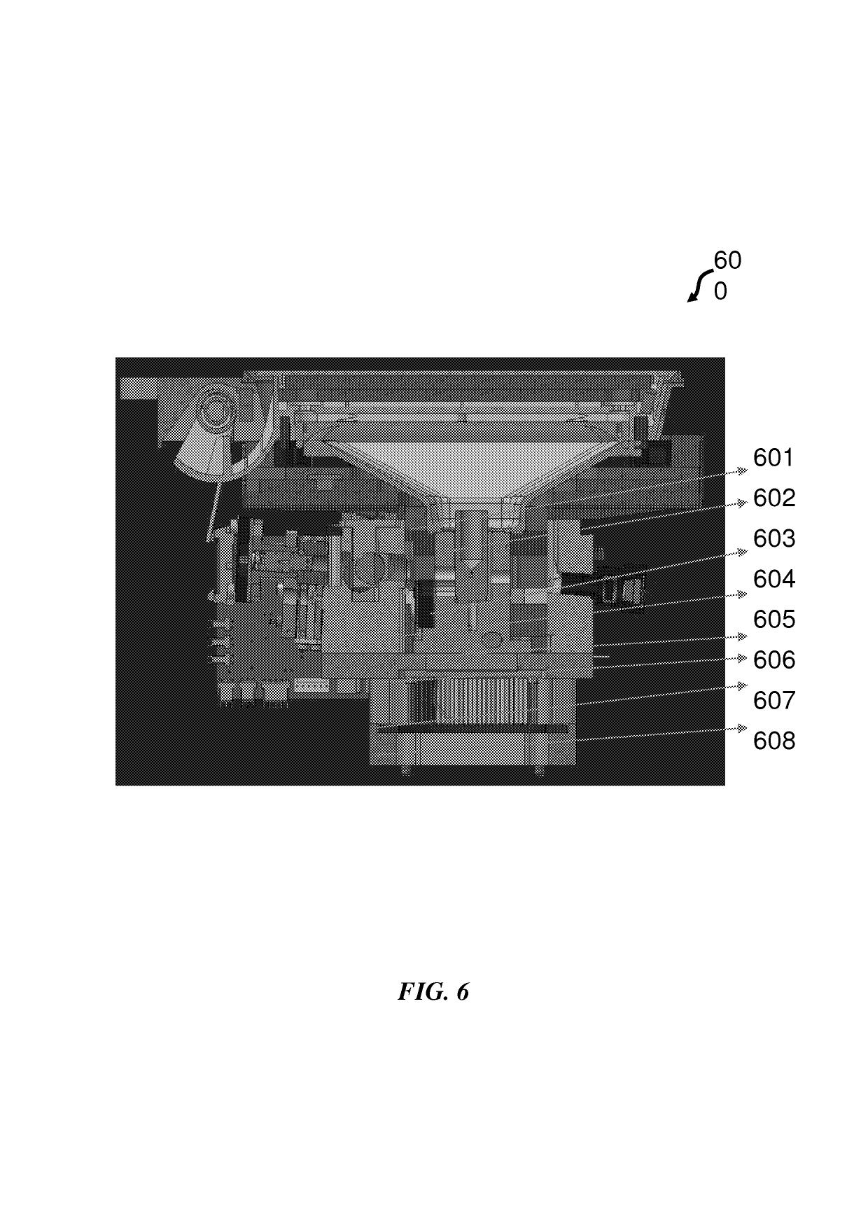

FIG. 6 is a cross-sectional view of an apparatus 600 that detects nanoparticle formation in fuels, in accordance with some embodiments. The apparatus 600 may include some or all components shown in the system 100 of FIG. 1 and may perform some or all of the methods shown in FIGS. 2A and 2B.

In some embodiments as shown, the apparatus 600 may include an air volume 601 on top of a sample cell 604, a metal funnel/ isothermal chamber 602, an insulating foam 603, a read head 605 about the sample cell 604, a nitrogen gas inlet 606, a Peltier element 607, or thermoelectric cooler, and a heatsink and exhaust fan 608. The apparatus 600 is constructed and arranged to lower a fuel cooling temperature to -50 ºC or less. The elements 601-608 are part of an optical bench for influencing cooling performance.

In some embodiments, the instrument 600 can operate at up to an 0ºC ambient temperature. That means, it can be placed in a cold room/freezer and push the sample temperature limit to -30 ºC or below with no hardware changes. In conventional instruments, on top of the sample cell there is a large metal funnel. When the lid is closed, it becomes an isothermal chamber and reduces the thermal gradients within the sample. However, due to the large thermal mass and big air volume on top of the sample, this design limits how fast and how much sample can be cooled. To address this, in some embodiments shown in FIG. 6, the metal funnel with an insulating plastic funnel 602 and fill air volume with insulating foam 603. This significantly reduces the overall thermal mass that needed to be cooled down. This should give additional 10C or more of cooling range.

In some embodiments, a conventional Peltier element can be replaced with a multi-stage Peltier cooling system 607-608.

Also, the nitrogen gas intake 606 may be used to keep the sample surrounding dry when operated at or below dew point. The system can inject cold nitrogen gas through the port, e.g., from a liquid nitrogen tank, using an insulating tubing. This will assist the Peltier cooling and can also improve the thermal ramp rates.

In some embodiments, the heatsink, exhaust fan 608, and insulation foam 603 can be upgraded for incremental increase in the range.

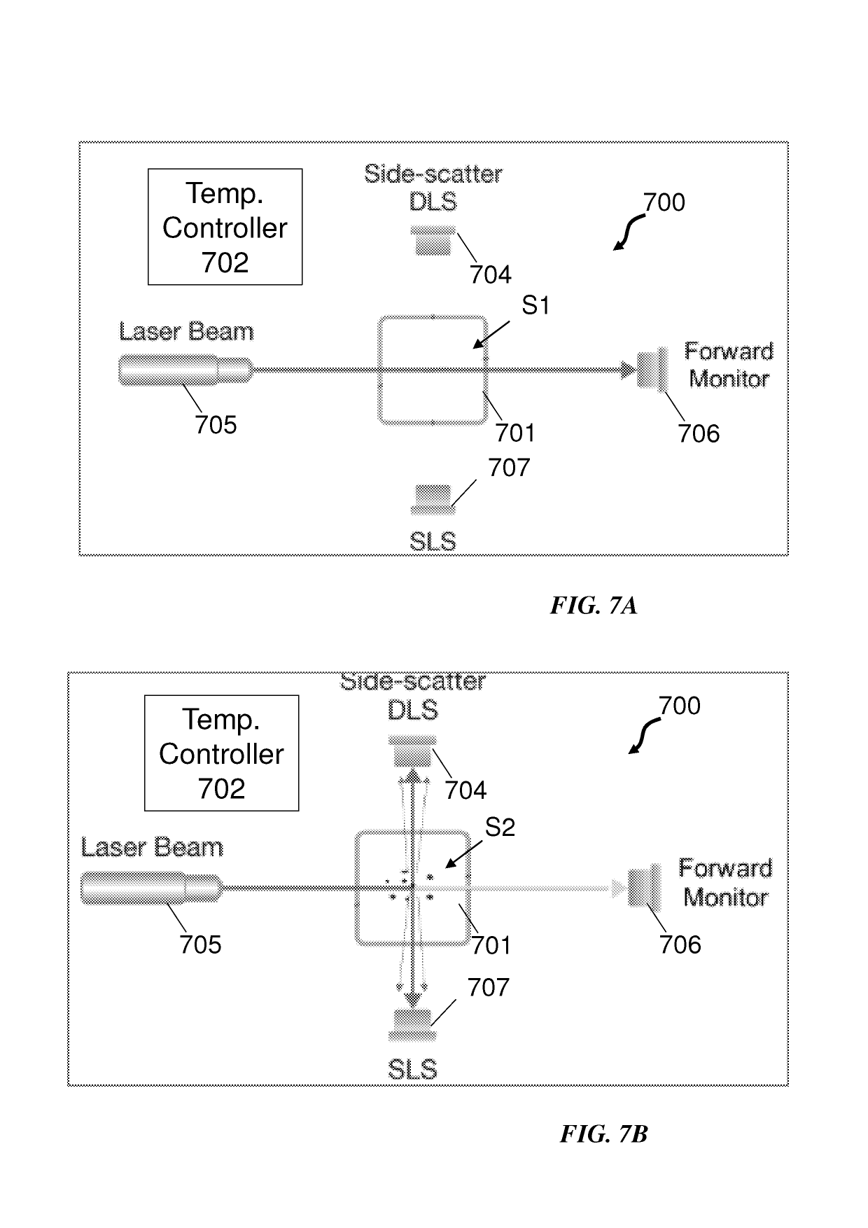

FIGS. 7A and 7B are block diagrams of a cloud point monitoring system 700, in accordance with other embodiments.

In some embodiments, the cloud point monitoring system 700 includes a light source 701, e.g., a laser, a temperature control system 702, a side-scattering DLS measurement system 704, and a forward monitor detector 706 similar to the components of the cloud point monitoring system 100. Details of these components are not repeated for brevity. In addition, the cloud point monitoring system 700 includes a static light scattering (SLS) measurement system 707. More specifically, the cloud point monitoring system 700 shown in FIG. 7A shows a clear fuel sample S1 in a sample cell 701 at a warmer temperature than the cloud point, with no particles. The cloud point monitoring system 700 shown in FIG. 7B shows a fuel sample S2 at a cooler temperature with particles present.

During operation, the light source 705 produces a laser beam focused into the sample cell 701 that contains the fuel S1, S2 (generally, sample S) under test. The forward monitor 706 monitors the intensity of ‘unscattered’ laser beam. When there are no particulates in the fuel under test (i.e., FIG. 7A), the forward monitor 706 measures a maximum intensity and is considered a baseline, or point at which the sample cell 701 is devoid of particles so that blank measurements can be taken of the sample absent particles. As the fuel cools down and particles are formed (transition from FIG. 7A to 7B), those particles start to scatter light in all directions and reduces the ‘unscattered’ intensity onto the forward monitor 706. So, a reduction in intensity at the forward monitor 706 can indicate the presence of particles and temperature at which that happens relates to cloud point of the fuel sample S.

In some embodiments, the SLS measurement system 707 is positioned to be 90 degrees on one side (but not limited thereto), and includes a photo detector or the like that measures static light scattering (SLS) signals. The SLS measurement system 707 measures a time averaged intensity that is scattered from the fuel S under test when the laser or other source of electromagnetic radiation output from the light source 705 interacts with the fuel. When there are no particulates, the SLS measurement system 707 reads a baseline intensity only from the fuel molecules. As the fuel cools down and particles are formed, those particles start to scatter light in all directions and the SLS signal starts to rise from the baseline and eventually saturates the detector 707. A rise in the SLS signal indicates the increased turbidity of the fuel. The temperature at which this occurs relates to the cloud point of the fuel.

At the opposite side of the cloud point monitoring system 700, namely, 90 degrees on the opposite side of the SLS measurement system 707 is the DLS measurement system 704 that includes a photo detector or the like that measures dynamic light scattering (DLS) signals. Here, the DLS measurement system 704 measures fast fluctuations in the scattered intensity. By looking at time correlation between these fluctuations, one can deduce the average size distribution of the particles in the fuel, down to 0.2nm. When there are no particulates, the DLS measurement system 704 reads baseline random fluctuations and reports no measurable time correlation and no reported size. As the fuel (S) cools down and particles are formed, those particles start to scatter light in all directions (shown in FIG. 7B) and the DLS measurement system 704 producing the DLS signal starts to see a higher time correlation in the measured intensity fluctuations and reports corresponding size of the particles.

FIG. 12 is a graph illustrating particle formation detection at the cloud point monitoring system of FIG. 11, in accordance with some embodiments.

Both the DLS measurement system 704 and SLS measurement system 707 include sensitive detectors with a high dynamic range. This allows the detectors to inform the user of the early detection of the cloud point well before the cloud point is reached as shown in FIG. 12. As shown, a fuel sample is cooled down at a constant ramp rate from a warm temperature, and is further monitored by the DLS measurement system 704 and SLS measurement system 707, including monitoring for particle size and FM readings. Both the particle size (shown in left y-coordinate as diameter nm) and SLS signal (shown in right y-coordinate) started to rise around 10.5 C, before the cloud point is reached. The forward monitor 706, on other hand, has a less sensitive detector than the DLS 704 and DLS 707 and can be used to measure the cloud point similar to the other standard test methods, for example, described in ASTM D5773 but not limited thereto.

With further regard to the foregoing, fuels are generally described. However, the foregoing embodiments may be used for other applications, which may arise that use fluids and particles other than wax particles and can be hard or soft particles, for example, lubricants, jet fuels, wax stocks, or the like.

What is claimed is:

1. A system for detecting a formation of nanoparticles in a fluid, comprising:
a temperature controller that provides a temperature of a source of fluid during a temperature ramp cool down operation;
a measurement system that detects a particle formation in the source of fluid at a wax particle formation temperature; and
an apparatus that detects a cloud point after the particle formation detection by the measurement system and at a temperature less than the wax particle formation temperature, wherein the wax particle formation temperature provides an early warning to users regarding the source of fluid before the fluid becomes visibly cloudy.

2. The system of claim 1, wherein the fluid includes a fuel.

3. The system of claim 1, wherein the particle formation includes an increase in size of nanoparticles of the particle formation during the temperature ramp cool down operation.

4. The system of claim 1, wherein the system measures a decrease in size of nanoparticles of the particle formation during a temperature ramp increase operation.

5. The system of claim 1, further comprising a plurality of sensors that measure the temperature of the source of fluid.

6. The system of claim 1, wherein the measurement system comprises a light source and a plurality of detectors that measure angles of light emitted from the light source and transmitting through the fluid to characterize the formation of nanoparticles of the fluid.

7. The system of claim 1, wherein the measurement system measures subvisible particles ranging from 0.5 nm to 2000nm.

8. The system of claim 1, wherein the temperature controller provides a constant temperature over a predetermined period of time.

9. The system of claim 1, wherein the temperature controller changes the temperature over a predetermined period of time.

10. The system of claim 1, where in the apparatus is a forward monitor.

11. The system of claim 1, wherein the measurement system includes at least one of a dynamic light scattering device and a static light scattering device comprising a photodiode.

12. A system for detecting a formation of nanoparticles in a fluid, comprising:
a temperature controller that provides a set temperature of a source of fluid for a predetermined period of time;
a monitoring system that performs continuous monitoring of a source of fluid at the set temperature over the predetermined period of time; and
a dynamic light scattering (DLS) measurement system that detects a formation of particles in the source of fluid at the set temperature during the predetermined period of time, wherein the monitoring system detects a cloud point after the particle formation detection by the DLS measurement system.

13. The system of claim 12, wherein the fluid includes a fuel.

14. The system of claim 12, wherein the particle formation includes an increase in size of nanoparticles of the particle formation during the temperature ramp cool down operation.

15. The system of claim 12, wherein the system measures a decrease in size of nanoparticles of the particle formation during a temperature ramp increase operation.

16. The system of claim 12, further comprising a plurality of sensors that measure the temperature of the source of fluid.

17. The system of claim 12, wherein the measurement system comprises a light source and a plurality of detectors that measure angles of light emitted from the light source and transmitting through the fluid to characterize the formation of nanoparticles of the fluid.

18. A system for detecting a formation of nanoparticles in a fluid, comprising:
a light source that generates a source of light at a fluid sample;
a temperature controller that provides a temperature of the fluid sample during a temperature ramp cool down operation;
a forward monitor that monitors an intensity of the source of light to indicate a presence of particles in the fluid sample and measures a cloud point;
a first photo detector that measures a static light scattering signal when the source of light interacts with the fluid sample;
a second photo detector that measures a dynamic light scattering signal to determine fluctuations in a scattered intensity when the particles scatter the source of light; and
a measurement system that detects a particle formation in the source of fluid at a wax particle formation temperature, wherein a combination of the first photo detector and the second photo detector detect a presence of the particles before the cloud point is reached.

19. The system of claim 18, wherein the particle formation includes an increase in size of nanoparticles of the particle formation during the temperature ramp cool down operation.

20. The system of claim 18, wherein the system measures a decrease in size of nanoparticles of the particle formation during a temperature ramp increase operation.
